(19) United States
(12) Patent Application Publication
(10) Pub. No.: US 2012/0220813 A1
(43) Pub. Date: Aug. 30, 2012

(54) DEVICES AND METHODS FOR APPLYING ENERGY TO A MUSCULAR LAYER

(76) Inventor: Sanford LANE, Sherborn, MA (US)

(21) Appl. No.: 13/357,823

(22) Filed: Jan. 25, 2012

Related U.S. Application Data
(60) Provisional application No. 61/435,879, filed on Jan. 25, 2011.

(51) Int. Cl. A61N 1/05; A61N 5/06; A61N 2/02; A61F 7/00

(52) U.S. Cl. 600/13; 607/133; 607/101; 607/92; 607/113

(57) ABSTRACT

A device for delivering energy to a muscle may include a housing and at least one energy conduit associated with the housing. The at least one energy conduit may be configured to facilitate transfer of energy from an energy source that may be configured to provide energy to muscle within an organ. The device may further include a plurality of needles connected to the at least one energy conduit. Each needle may be configured to pierce tissue. The plurality of needles may include at least one needle configured to deliver energy through an energy emitting portion. The at least one needle may be sized so that when inserted into a wall of the organ, the energy emitting portion may be located within a muscle layer, and such that a majority of energy delivered via the at least one needle may be substantially confined to the muscle layer.

### CROSS-REFERENCE TO RELATED APPLICATION

This applications claims the benefits of priority under 35 U.S.C. §§119-120 to U.S. Provisional Application 61/435,879, filed on Jan. 25, 2011, the entire disclosure of which is incorporated herein by reference.

### TECHNICAL FIELD

Embodiments of the present disclosure generally relate to applying energy to a muscular layer. For example, aspects of the present disclosure may be used to apply energy to a stomach muscle to reduce stomach volume and/or cause a satiating effect.

### TECHNICAL BACKGROUND

Obesity can lead to a variety of health problems, including, but not limited to heart disease, stroke, diabetes, and pulmonary disease. Therefore, there may be a need for mechanisms to treat obesity. In addition, even in individuals who are not obese, weight control may be a challenge, and those individuals may desire mechanisms to control appetite.

A variety of therapies have been used to reduce the volume of the stomach to achieve these ends. For example, gastric bypass surgery involves dividing the stomach into two, isolated portions, with one of the portions having a reduced food capacity, and connecting that portion to the jejunum through a small stoma, which reduces the size of the stomach. An alternative procedure involves placing a band around a portion of the gastrointestinal (GI) tract to constrict flow to the stomach.

A further alternative procedure involves application of heat to the stomach wall to reduce stomach volume, although such an approach may suffer from limited effectiveness and/or tissue damage whose harm may outweigh the benefit achieved. There is, accordingly, a need for devices and methods that may allow for effective weight control, while reducing any risks associated with the foregoing procedures.

Although the foregoing background is discussed in connection with a stomach, embodiments of the invention, as summarized, illustrated, described and claimed herein, may be applied more broadly to body parts other than the stomach.

### SUMMARY

Embodiments of the present disclosure may include a device for delivering energy to a muscle. The device may include a housing, and at least one energy conduit associated with the housing. The at least one energy conduit may be configured to facilitate transfer of energy from an energy source that may be configured to provide energy to muscle within an organ. The device may further include a plurality of needles connected to the at least one energy conduit, each needle may have a proximal end, a distal end, and a tip configured to pierce tissue. The plurality of needles may include a first group having at least one needle configured to deliver energy through an energy emitting portion that may be spaced from the proximal end of the at least one needle. The at least one needle of the first group may be sized so that when inserted into a wall of the organ, the energy emitting portion may be located within a muscle layer, and the energy emitting portion may be configured such that a majority of energy delivered via the at least one needle of the first group may be substantially confined to the muscle layer.

By way of example only, in various embodiments, the device for delivering energy to the muscle layer may include one or more of the following features: the plurality of needles may be configured to transition between a first configuration in which the tip of each needle is shielded, and a second configuration in which at least the tip of each needle is exposed for insertion into the wall of the organ; the plurality of needles may be movable relative to the housing such that in the first configuration the tip of each needle may be shielded within the housing, and in the second configuration at least the tip of each needle may extend from a distal end of the housing; and the device may include a sheath, wherein the plurality of needles may be fixed relative to the housing, such that in the first configuration the tip of each needled is shielded by the sheath, and in the second configuration the sheath is retracted proximally of at least the tip of each needle.

In addition, the housing may be configured for deployment with at least one of a laparoscope and an endoscope; the energy emitting portion of the at least one needle in the first group may include the needle tip; the plurality of needles may include a second group having at least one needle such that the first group may be configured to serve an anode function and the second group may be configured to serve a cathode function; the energy source is an RF energy source; the housing may be configured to engage a stomach wall; the housing and needles may be sized for delivery to an interior of a stomach wall via an esophagus; the housing and needles may be sized for delivery to an exterior of a stomach wall via a laparoscopic incision; the plurality of needles may be configured to cause a satiated sensation in a patient after energy is delivered via the energy emitting portion; and the at least one needle in the first group may include a portion that may be at least partially shielded to prevent energy emission from the portion.

Embodiments of the present disclosure may further include a method of reducing a hunger sensation. The method may include inserting at least one first needle into a muscle layer of a stomach wall, and the needle may have an energy emitting portion; delivering energy from an energy source to the needle; and emitting energy from the energy emitting portion into the muscle layer for a period of time sufficient to contract muscle in the muscle layer.

In various embodiments, the method may include one or more of the following features: controlling energy emission such that substantially all energy emitted may be delivered to the muscle layer; inserting at least a second needle into the muscle layer of the stomach wall, the at least one first needle may be configured to serve an anode function, and the at least second needle may be configured to serve a cathode function; transitioning the at least one needle from a first configuration in which a tip of the at least one needle may be shielded to a second configuration in which the tip of the at least one needle may be unshielded, prior to inserting the at least one needle into the muscle layer of the stomach wall; transitioning the at least one needle may include at least one of moving the at least one needle relative to a housing and retracting a sheath; and the housing and needles may be sized for delivery via at least one of an esophagus and a laparoscopic incision.

Embodiments of the present disclosure may also include a device for reducing a hunger sensation. The device may include at least one controller that may be configured to control energy delivery to a plurality of needles. The needles may be configured to deliver energy from a location within a muscle layer of a stomach wall. In addition, the at least one controller may be configured to cooperate with the plurality of needles to regulate energy delivery in a manner such that the energy delivered via the plurality of needles may be substantially confined at the muscle layer.

Additional explanations of aspects of the disclosure will be set forth in part in the description which follows, and in part will be understood from the description, or may be learned by practice of the invention. It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the invention and together with the description, serve to explain inventive principles.

### BRIEF DESCRIPTION OF THE DRAWINGS

FIG. 1 is a perspective view of an energy delivery device according to a first exemplary embodiment of the present disclosure.

FIG. 2A is an enlarged view of the head of the energy delivery device of FIG. 1.

FIG. 2B is an end view of the head of the energy delivery device of FIG. 1.

FIGS. 3A-3B are perspective and end views of alternative energy delivery device according to a second exemplary embodiment of the present disclosure.

FIG. 4A is a perspective view of an energy delivery device according to a third exemplary embodiment of the present disclosure.

FIG. 4B is a perspective view of an energy delivery device according to a fourth exemplary embodiment of the present disclosure.

FIGS. 4C-4D are perspective views of the energy delivery device of FIG. 4A with a selectively movable sheath in first and second configurations, respectively.

FIGS. 5A-5B are perspective views of an energy delivery device according to a fifth exemplary embodiment of the present disclosure.

FIGS. 6A-6B are perspective views of a needle consistent with exemplary embodiments of the present disclosure.

FIGS. 7A-7B are perspective views of the energy delivery device of FIG. 1 operatively place against a stomach.

FIGS. 8A-8B are perspective views of layers of the stomach wall with the needle of FIG. 6A there through.

### DESCRIPTION OF EXEMPLARY EMBODIMENTS

Reference will now be made in detail to a few exemplary embodiments of the present disclosure, examples of which are illustrated in the accompanying drawings. Wherever possible, the same reference numbers will be used throughout the drawings to refer to the same or like parts.

Embodiments of the present disclosure relate generally to devices and methods for applying energy to a muscle layer. Some embodiments of the present disclosure may be used in connection with the application of energy to muscle in a stomach wall in order to reduce stomach volume or cause a satiation effect. It should be emphasized, however, that embodiments of the present disclosure may also be used in other medical procedures where application of energy may be desired.

In accordance with embodiments of the present disclosure, there may be provided a device for delivering energy to a muscle including a housing. A housing may include any physical structure capable of retaining structures including, but not limited to, a plurality of needles. In some embodiments the housing may be configured for use within a human or animal body, and may be of any suitable shape and/or size such that the housing may be configured for deployment within at least one of a laparoscope and an endoscope. For example, the housing may be sized for delivery to an interior of a stomach wall via an esophagus. In addition, or alternatively, the housing may be sized for delivery to an exterior of a stomach wall via a laparoscopic incision. When configured for use within a living organism, the housing may be constructed of a biocompatible material. Suitable biocompatible materials may include, but are not limited to, polymers, metals, metal alloys, ceramics, or combinations thereof.

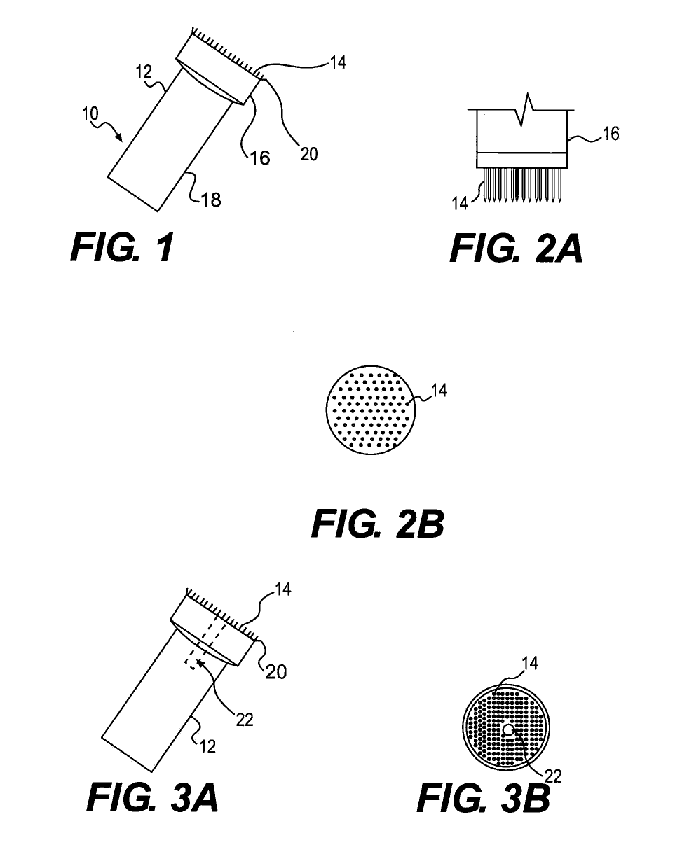

FIG. 1 illustrates, in schematic form, one example of a housing 12 in connection with a device 10 for delivering energy to a muscle, according to a first embodiment of the present disclosure. The housing may have a proximal end and a distal end. For purposes of this disclosure, “proximal” refers to the end closer to the device operator during use, and “distal” refers to the end further from the device operator during use.

As illustrated in FIG. 1, housing may include a shaft 18 and a head 16 on the distal end of the shaft 18. The shaft may be of any suitable shape and configuration for supporting the head, such as, for example, a hollow longitudinal member. The shaft may include any cross-sectional shape. For example, as shown in FIG. 1, shaft 18 may be cylindrical, having a circular cross-section. In other embodiments, however, shaft may have any desired cross-sectional shape.

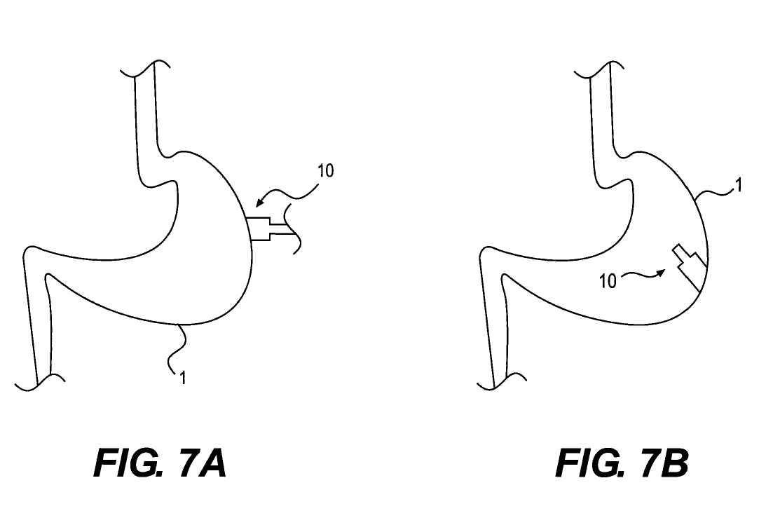

The head of the housing may extend distally from the distal end of the shaft. The head may further include the distal-most surface of the housing, and may be configured to project and/or support a plurality of needles. The head may further have any size and cross-sectional shape so long as the head may be configured to engage tissue of a patient. For example, as shown in FIGS. 7A and 7B, the head may include any size and/or shape such that the housing may be configured to engage a stomach wall.

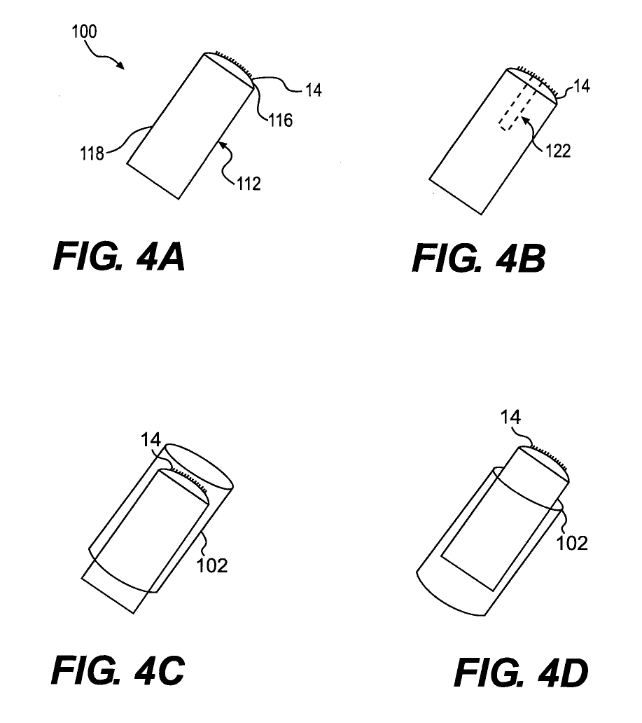

FIG. 1 illustrates an embodiment where head 16 may have a diameter larger than that of the shaft 18. Alternatively, in another embodiment, as illustrated in FIG. 4A, head 116 may have a diameter substantially similar to that of the shaft 118. In addition, in one embodiment, as shown in FIG. 1, the head 16 may include a circular cross-sectional shape and may have a flat or concave distal surface. Alternatively, as illustrated in FIG. 4A, the head 116 may include a circular cross-sectional shape with a convex distal surface.

In accordance with embodiments of the invention, there may be provided at least one energy conduit associated with the housing, the at least one energy conduit configured to facilitate transfer of energy from an energy source, the energy source configured to provide energy to muscle within an organ. In some embodiments, the energy conduit may be located in or on the housing. The energy conduit may include one or more conductive wires, fibers, or other channels for conveying energy from the source via the housing.

Embodiments of the invention, in their broadest sense, may not be limited to a particular source of energy. Examples of energy sources that may be used in connection with the invention may include, but are not limited to, light sources such as lasers; electromagnetic sources such as microwave, infrared and radio frequency; thermal conductive, and sound energy, such as ultrasound. The foregoing energy sources may be applied, for example, in a manner to cause a level of heat within a muscle layer which may result in damage to muscle tissue, and ultimately, micro scarring during a healing process.

In one embodiment, for example, the energy source may be a radiofrequency (RF) energy source. In such instance, the housing may support a bipolar energy deliver mechanism. For example, one or more needles may include a portion that transmits and another portion that receives. In one embodiment, a plurality of needles may include a first group of at least one needle and a second group of at least one needle. The at least one needle of the first group may include an energy emitting portion. The at least one needle of the second group may include an energy receiving portion. Accordingly, the first group may be configured to serve an anode function and the second group may be configured to serve a cathode function. The functionality of the first and second groups may change. For example during one time period, the first group may serve as an anode and the second group may serve as the cathode. During another time period, the functions may be reversed. This may occur, for example, as the result of a controller associated with the energy source.

The at least one energy conduit may further be associated with a plurality of needles in order to facility transfer of energy to the muscle with an organ. In one embodiment, for example, the plurality of needles may be connected to an energy source via the at least one energy conduit. Each needle may further be configured to extend from the head of the housing of the device, such that each needle may form an array of needles extending distally from the housing. The plurality of needles may include at least two needles. FIGS. 2B and 3B illustrate an end view of the housing 12, 112, showing an example of array of needles 14 dispersed on the head of the housing.

Each needle may have a proximal end, a distal end, and a tip configured to pierce tissue. Each needle may further include any suitable size and/or shape so long as at least the tip may be configured to extend to the muscle layer of an organ. For example, in one embodiment, the needle may have a length suitable for the tip to extend to a depth between approximately 1 mm and 5 mm within an organ. Each needle may further be sized and shaped for delivery to an interior of a stomach wall via an esophagus and/or for delivery to an exterior of a stomach wall via a laparoscopic incision.

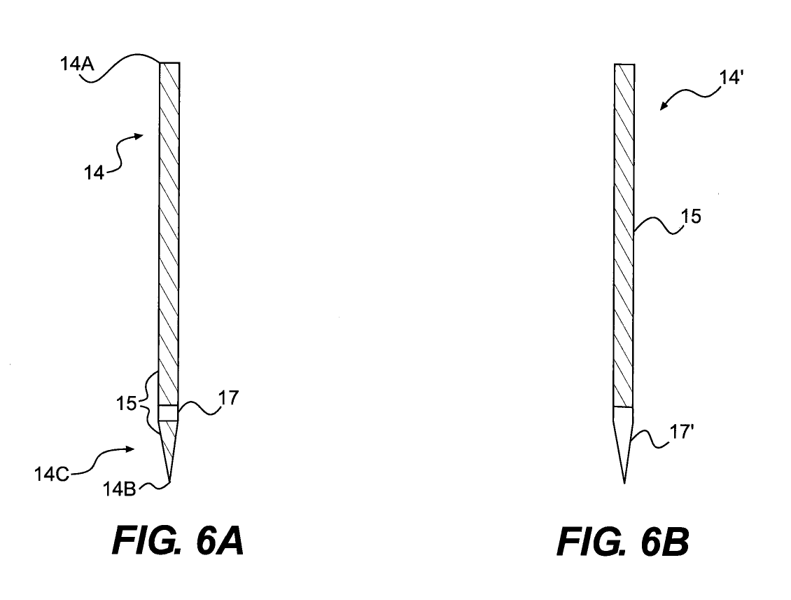

As previously discussed, the plurality of needles may further include a first group having at least one needle that may be configured to deliver energy through an energy emitting portion. The energy emitting portion may extend along the entire length of each needle. Alternatively, as illustrated in the embodiments of FIGS. 6A and 6B, the energy emitting portion 17, 17′ may be confined to a tip region of each needle. This can be achieved, for example, by shielding the needle in a manner that permits energy to exit only through the tip regions. Alternatively, the needles may be configured so that energy is emitted from a region intermediate the proximal and distal ends of the needle.

In the embodiment of FIG. 6A, for example, the energy emitting portion 17 may be located between the proximal end 14a and distal end 14b of the needle. Alternatively, the embodiment of FIG. 6B illustrates that the energy emitting portion 17′ of the at least one needle in the first group may include the needle tip 14b.

The energy emitting portion may be formed of any suitable biocompatible, conductive material. Suitable biocompatible conductive materials may include, but are not limited to, aluminum, stainless steel, silver, gold, metal alloys, and combinations thereof. Depending on energy source, the energy emitting portion may include components such as optical fiber or other suitably conductive or transmissive materials.

In some embodiments, the at least one needle in the first group may include a portion that may be at least partially shielded to prevent energy emission from the portion. Accordingly, the shield may include a non-conductive biocompatible material, including, but not limited to carbon fiber, fiberglass, or plastic. Each needle may, therefore, be rigid, semi-rigid, or flexible, so long as each needle may be configured to reach a muscle layer when inserted through the wall of an organ. Each needle may further be solid or hollow. In one embodiment, for example, the needle may be hollow such that an electrode may pass through the needle and reach the energy emitting portion for application of energy to tissue.

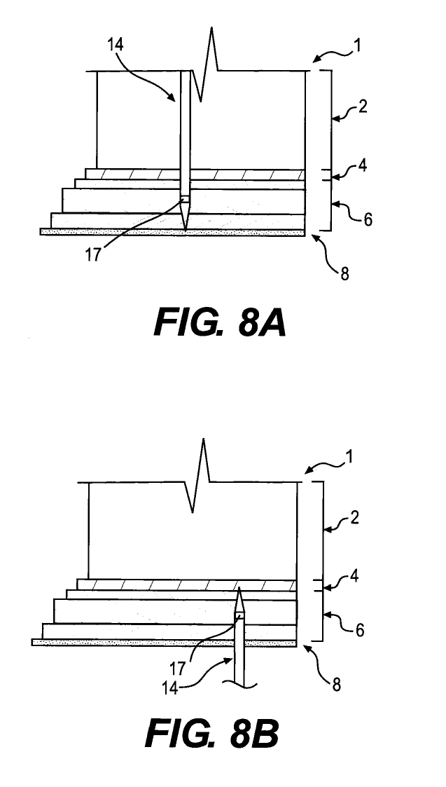

The shield may include one or more segments partially or completely surrounding the needle, such that the energy emitting portion may be located between the proximal and distal ends of the needle. For example, the embodiment of FIG. 6A illustrates that the shield 15 may include two segments with a single energy emitting portion 17 located there between. In alternative embodiments the shield may include more than two segments, and there may be multiple energy emitting portions located on the needle. In addition, in alternative embodiments, the energy emitting portion 17′ may include the tip 14C of the needle 14′, as illustrated in the embodiment of FIG. 6B. The energy emitting portion may, accordingly, be located at any desired location along the length of the needle. For example, as illustrated in FIGS. 8A-8B, the at least one needle of the first group may be sized so that when inserted into a wall of an organ, the energy emitting portion may be located within the muscle layer of the organ.

FIGS. 8A-8B further illustrate that, if the organ is a stomach, the at least one needle 14 may be sized such that the energy emitting portion 17 may be located within the muscle layer 6, regardless of the delivery path of the device. For example, as illustrated in FIG. 8B, if the device is inserted laparoscopically, the needle 14 may pierce through the serosa layer 8 of the stomach prior to reaching the muscle layer 6. Alternatively, FIG. 8A illustrates that if the device is inserted endoscopically, the needle may pierce through the mucosa 2 and submucosa 4 layers prior to reaching the muscle layer 6.

The energy emitting portion may further be sized and configured such that a majority of energy delivered via the at least one needle of the first group may be substantially confined to the muscle layer. This may be accomplished as the result of a needle design where the energy emitting portions of the needles are spaced so as to reside within the muscle when the needle is fully/properly deployed. For example, using the known anatomy of the typical stomach, it may be possible to size the needles and the energy emitting portions thereof so that when the needles in the housing are applied with uniform pressure to the wall of the stomach, the energy emitting portion of each needle may substantially reside within the muscle layer.

To achieve this result, the housing may include a mechanism for applying uniform pressure to the portion of the organ contacting the device for delivering energy. In one embodiment, this function may be achieved by fixing the needles to the housing such that the housing acts as a stop control the depth to which the needles are permitted to travel. In this embodiment, a user will press the housing against the organ until the housing moves no further, thus ensuring uniform and proper needle depth penetration.

In an alternative embodiment, the needles may be biased, such as through spring loading. After the housing is pressed against the organ, a trigger may be released, causing the needles to pierce the organ to a predetermined depth.

In order to maintain a uniform pressure throughout a procedure, an operator may continue to exert a force on the head 16. Alternatively, or in addition, the housing may be configured to include a vacuum chamber to hold the head 16 against the organ using vacuum pressure. In this embodiment, illustrated in FIG. 1, for example, a vacuum seal 20 may extend distally from the head 16 of the housing 12. The vacuum seal 20 may be configured to maintain a vacuum when the housing engages with the wall of the organ.

It may be beneficial, in some embodiments, to prevent the needles from engaging with tissue before such time as the needles are in the vicinity of the organ to be treated. To this end, each of the plurality of needles may further be configured to transition between a first configuration in which the tip of each needle may be shielded, and a second configuration in which at least the tip of each needle can be inserted into the wall of an organ. In one embodiment, for example, the needles may be movable relative to the housing such that in the first configuration the tip of each needle may be shielded within the housing, and in the second configuration at least the tip of each needle may extend distally from the housing. In a further embodiment, the device may include a sheath. The sheath may be a retractable and may be configured to shield the tip of each needle as the needle is inserted into a patient and travels to the site for receiving energy. In this embodiment, the plurality of needles may be fixed relative to the housing, such that in the first configuration the tip of each needle is shielded by the sheath, and in the second configuration the sheath may be retracted proximally of at least the tip of each needle. For example, FIG. 4C illustrates the first configuration, where needle tips 14 are shielded by movable sheath 102, and FIG. 4D illustrates the second configuration, where the sheath 102 is proximally retracted and needle tips 14 are exposed.

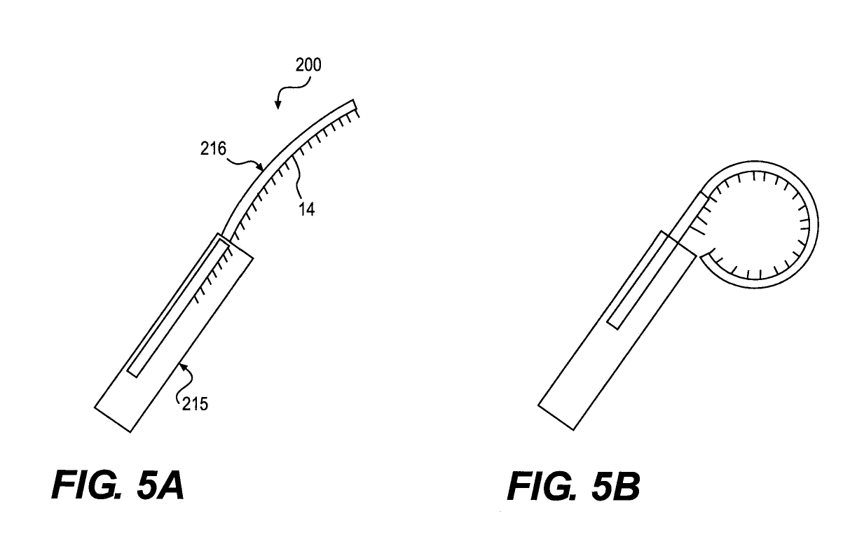

The sheath may also be configured for use with the devices having a housing configuration as illustrated in FIGS. 1, 3A. In an alternative embodiment, the sheath may be configured for use with the device illustrated in FIGS. 5A and 5B. In this alternative embodiment, the device 200 for delivering energy may include a flexible elongate band 216. As illustrated in FIG. 5B, the flexible elongate band 216 may be configured to contract and form a collar-like structure, which may be used to treat a smaller area of an organ and/or treat an area of an organ in a ring-like manner. Similar to the embodiments of FIGS. 1, 3A, and 4A-B, the device 216 of FIGS. 5A-B may include a plurality of projecting needles 14. The plurality of needles may be shielded by a sheath 212 during delivery of the device to the desired energy delivery sight. The sheath 212 may further be retracted to allow the contraction of the device 200.

FIGS. 4C-4D and 5A-5B illustrate embodiments of sheaths that include a single, hollow member surrounding the device for delivering energy. Alternatively, or in addition to, sheath may include a plurality of tip covers, where each tip cover may enclose a tip of a respective needle. Each tip cover may, therefore, be configured to selectively enclose a respective needle tip in a first configuration, and retract in a second configuration to allow for at least the tip of each needle to protrude from the tip cover. Each tip cover may further be any suitable shape or size so long as it may be configured to selectively enclose and retract around a needle tip. In one embodiment, for example, each tip cover may include a ball-like shape.

Application of energy to muscle may cause therapeutic responses. For example, the energy may cause damage to tissue, and the healing that subsequently occurs may have a therapeutic effect. By choosing a predetermined size and needle orientation as described earlier, it may be possible to substantially confine energy application to desired region, while substantially avoiding energy applications to other regions. For example, if energy is substantially confined to a muscle layer, substantial damage to surrounding non-muscle tissue may be avoided. While some collateral damage to surrounding tissue may occur when using embodiments of the invention, the damage may not be sufficiently substantial so as to negate the positive therapeutic benefits of the invention. Alternatively, or in addition to purposeful causation of damage to tissue, energy application may cause muscle to contract, which may be useful to achieve a desired effect.

The organ to which energy may be applied may be any organ where muscle contraction may be desired. In one embodiment, as illustrated in FIGS. 7A and 7B, the organ may be a stomach 1. FIGS. 8A and 8B illustrates that the stomach 1 includes a plurality of layers. Energy may be emitted to any layer where contraction is desired. In one embodiment, as illustrated in FIGS. 8A and 8B, the energy emitting portion 17 of the needle 14 may be confined to the muscle layer 6. Energy delivery may cause contraction of the muscle layer in the stomach, which may thereby reduce the volume of the stomach. In addition, the contraction of the muscle layer of the stomach may further cause a reduction of hunger in the patient.

A reduction in a hunger sensation in a patient may occur as a result of the wound healing response to damage purposefully caused by the application of energy. Energy application to the muscle layer of the stomach may create a matrix of scar tissue within the muscle layer. The scar tissue may result in a patient's inability to expand the stomach in response to food intake. The stomach's inability or reduced ability to expand may, in turn, cause neurological signals to be sent to the brain, which may cause a satiated sensation in the patient. Accordingly, the plurality of needles may be configured to cause a degree of damage to stomach muscle sufficient to cause a satiated sensation in a patient after energy is delivered via the energy emitting portion. The reduction in hunger sensation may begin to occur shortly after treatment and/or may first occur after a sufficient period of time has elapsed to permit the healing process to begin.

In one embodiment, for example, a method for reducing a hunger sensation in a patient may first include inserting one or more of a previously described needle into a muscle layer of a stomach wall and emitting energy from the energy emitting portion into the muscle layer for a period of time sufficient to contract muscle in the muscle layer.

Thus, a device for delivering energy may be placed at the wall of an organ, such as a stomach, at a location selected where tissue (e.g., muscle) may be contracted. For example, FIGS. 7A and 7B illustrate that the device may be placed on the interior or exterior of a stomach wall. Accordingly, the device may be sized for delivery via at least one of an esophagus and a laparoscopic incision of a patient. Alternatively, in other embodiments of the invention, a device consistent with the invention may be larger, for use in more invasive surgical procedures. The device may further be placed against the wall of the stomach at any angle known to those skilled in the art that that may be advantageous to the targeted delivery of energy. In one embodiment, for example, the device may be placed such that the needles may be approximately perpendicular to the wall of the stomach, as illustrated in FIGS. 8A and 8B.

Prior to the insertion of the at least one first needle into the muscle layer of the stomach wall, the method may include transitioning the needle from a first configuration in which a tip of the at least one first needle may be shielded to a second configuration in which the tip of the at least one first needle may be unshielded. The transitioning may occur by moving the at least one first needle distally relative to a housing in which the needle may be shielded. In addition, or alternatively, the transitioning between the first and second configurations may occur by retracting a sheath proximally relative to a distal end of the at least one first needle.

Consistent with the invention, energy may be delivered in a variety of ways. For example, in one embodiment, a method may include delivering energy in a bipolar manner such that at least two needles are used. At least one needle may be configured to serve an anode function, and at least a second needle may be configured to serve a cathode function. In some embodiments, there may be an array of cathode needles and an array of anode needles. In other embodiments, a group of needles may at differing times serve cathode and anode functions. This may be accomplished through a controller associated with the energy sources. Although not necessarily required, energy transfer may be facilitated through the use of an electrolyte solution or other material for enhancing energy transfer between energy emitting portions and tissue.

In an alternative embodiment, an energy emitting portion of a device for delivering energy may be inserted into a first layer of tissue and may be configured to deliver energy in a manner that may target a second layer of tissue without affecting the first layer of tissue. For example, in an embodiment of a method for reducing hunger in a patient, an energy emitting portion may be inserted into a serosa layer of a stomach wall. Energy may then be emitted from the energy emitting portion such that the serosa layer may not be affected by the emitted energy. Alternatively, the energy emitting portion may be inserted into the mucosa or submucosa layer of the stomach wall, where the energy emitting portion may reside during energy emission.

Energy emission directed to the second layer of tissue that may not affect the first layer of tissue in which the energy emitting source resides may be achieved by applying a source of energy at a predetermined wavelength and/or frequency such that the energy source may specifically target the second layer of tissue. For example, in one embodiment, the energy source may be a laser, and a predetermined wavelength may be chosen that may allow for direct targeting of the muscle tissue. In another embodiment, the energy source may be an RF energy source, and a predetermined frequency wavelength may be chosen. In further embodiments, the energy source may be an ultrasound or any other suitable energy source known to those skilled in the art that may be configured to be delivered in a manner that may allow for specific targeting of a tissue layer.

The period of time of energy delivery that may be sufficient to contract muscle and or cause therapeutic tissue damage may depend on several factors, including, but not limited to the power, frequency, amplitude, pulse configuration, and pulse width of the energy applied. Energy power may be applied in a range of approximately 1 mwatts to 200 watts. The pulse configuration of the energy delivered may be in the range of approximately 1 to 1000 pulses per second. In addition, the pulse width may be in the range of approximately 1 ms to 10 seconds, and the frequency of energy delivered may be in the range of approximately 100 KHz to 5 MHz. The period of time of energy delivery may further depend on the extent of the treatment. The extent of the treatment may include, but is not limited to, the amount of tissue treated and the extent of the tissue contraction.

After energy is delivered to the muscle layer of a stomach for the sufficient amount of time, the energy may be turned off, the needles may be retracted from the stomach wall, and the device may be transitioned to another location on the stomach wall such that the device may emit energy to contract the muscle at another desired location. This may be repeated until each desired area of the stomach has been contracted to cause the desired amount of volume reduction of the patient's stomach. Consistent with some methods, the totality of the treatment region may take the form of a closed or open circumferentially extending band around the stomach. In other methods, select regions of the stomach may be selected for treatment, depending on the desire of the treating physician.

Achievement of the desired extent of muscle contraction at each location may be determined by a feedback control mean mechanism. Accordingly, sensors or visualization devices may allow for a device operator to determine that a sufficient amount of energy has been delivered to the muscle layer at the desired location. The sensors or visualization devices may also allow a device operator to impose measures for preventing unwanted tissue damage.

Such a feedback control mechanism may be internal or external to the housing. Alternatively, the feedback control means may not be attached to the housing at all. In one embodiment, for example, the feedback control mechanism may be located on another portion of the device, such as on at least one of a plurality of needles. In a further embodiment, the feedback control means may not be attached to the device and may be inserted through a separate lumen in an endoscope or a laparoscopic incision different from that where the device may be inserted. Accordingly, the feedback control mechanism may include, but is not limited to visualization devices and sensors for sensing impedance and/or temperature levels of the tissue receiving energy.

The embodiments of FIGS. 3B and 4B illustrate one example of a feedback control mechanism in the form of a temperature sensor 22, 122 in the housing 12, 112. The temperature sensor may be any suitable sensor known to those skilled in the art configured to sense the temperature of the tissue that may contact the device for delivering energy. Accordingly, temperature sensor may include, but is not limited to an infrared sensor, thermistors, a semi-conductor, a non-contact infrared detector, and/or a fiber-optic sensor.

To prevent unwanted tissue damage, a temperature control mechanism may optionally be employed. For example, in one embodiment, the temperature control mechanism may include a cooling lumen, such that cooling sources, including, but not limited to, fluid or gas, may be applied to the treated tissue. This may result in temperature control of the tissue or prevent unwanted results to the tissue due to overheat application.

In one embodiment, control of the device for delivering energy may be accomplished directly by the device operator. In addition, or alternatively, control of the device for delivering energy may be accomplished through the use of a controller operatively attached to the device. The controller may be a processor programmed to delivery energy to tissue. Accordingly, the controller may be configured to control energy delivery to a plurality of needles, with the needles being configured to delivery energy from a location within a muscle layer of a stomach wall. The controller may also be configured to cooperate with the plurality of needles to regulate energy delivery in a manner such that the energy delivered via the plurality of needles may be substantially confined to the muscle layer. By way of example, the controller may be programmed with thresholds of energy delivery to cause a desired amount of tissue damage without causing undesirable damage that negates the value of the treatment. Moreover, the controller may be configured to control the amount of pressure uniformly applied to the wall of the stomach by the device, such that a homogenous distribution of energy may be achieved within the muscle layer.

Other embodiments of the present disclosure will be apparent to those skilled in the art from consideration of the specification and practice of the invention disclosed herein. It is intended that the specification and examples be considered as exemplary only, with a true scope and spirit of the present disclosure being indicated by the following claims.

What is claimed is:

1. A device for delivering energy to a muscle, comprising:
a housing;
at least one energy conduit associated with the housing, the at least one energy conduit configured to facilitate transfer of energy from an energy source, the energy source configured to provide energy to muscle within an organ; and
a plurality of needles connected to the at least one energy conduit, each needle having a proximal end, a distal end, and a tip configured to pierce tissue, wherein the plurality of needles includes a first group having at least one needle configured to deliver energy through an energy emitting portion, the energy emitting portion being spaced from the proximal end of the at least one needle, wherein the at least one needle of the first group is sized so that when inserted into a wall of the organ, the energy emitting portion is located within a muscle layer, and wherein the energy emitting portion is configured such that a majority of energy delivered via the at least one needle of the first group is substantially confined to the muscle layer.

2. The device of claim 1, wherein the plurality of needles are configured to transition between a first configuration in which the tip of each needle is shielded, and a second configuration in which at least the tip of each needle is exposed for insertion into the wall of the organ.

3. The device of claim 2, wherein the plurality of needles are movable relative to the housing such that in the first configuration the tip of each needle is shielded within the housing, and in the second configuration at least the tip of each needle extends from a distal end of the housing.

4. The device of claim 2, further comprising a sheath, wherein the plurality of needles are fixed relative to the housing, such that in the first configuration the tip of each needled is shielded by the sheath, and in the second configuration the sheath is retracted proximally of at least the tip of each needle.

5. The device of claim 1, wherein the housing is configured for deployment with at least one of a laparoscope and an endoscope.

6. The device of claim 1, wherein the energy emitting portion of the at least one needle in the first group includes the needle tip.

7. The device of claim 1, wherein the plurality of needles includes a second group having at least one needle such that the first group is configured to serve an anode function and the second group is configured to serve a cathode function.

8. The device of claim 1, wherein the energy source is an RF energy source.

9. The device of claim 1, wherein the energy source includes at least one of a light source, an electromagnetic source, an infrared source, a radio frequency source, a thermal conductive source, and a sound energy source.

10. The device of claim 1, wherein the housing is configured to engage a stomach wall.

11. The device of claim 1, wherein the housing and needles are sized for delivery to an interior of a stomach wall via an esophagus.

12. The device of claim 1, wherein the housing and needles are sized for delivery to an exterior of a stomach wall via a laparoscopic incision.

13. The device of claim 1, wherein the plurality of needles are configured to cause a satiated sensation in a patient after energy is delivered via the energy emitting portion.

14. The device of claim 1, wherein the at least one needle in the first group includes a portion that is at least partially shielded to prevent energy emission from the portion.

15. A method of reducing a hunger sensation, comprising:
inserting at least one first needle into a muscle layer of a stomach wall, the needle having an energy emitting portion;
delivering energy from an energy source to the needle;
emitting energy from the energy emitting portion into the muscle layer for a period of time sufficient to contract muscle in the muscle layer.

16. The method of claim 15, further comprising controlling energy emission such that substantially all energy emitted is delivered to the muscle layer.

17. The method of claim 15, further comprising inserting at least a second needle into the muscle layer of the stomach wall, the at least one first needle being configured to serve an anode function, and the at least second needle being configured to serve a cathode function.

18. The method of claim 15, further comprising transitioning the at least one needle from a first configuration in which a tip of the at least one needle is shielded to a second configuration in which the tip of the at least one needle is unshielded prior to inserting the at least one needle into the muscle layer of the stomach wall.

19. The method of claim 18, wherein transitioning the at least one needle includes at least one of moving the at least one needle relative to a housing and retracting a sheath.

20. The method of claim 19, wherein the housing and needles are sized for delivery via at least one of an esophagus and a laparoscopic incision.

21. A device for reducing a hunger sensation, comprising:
at least one controller configured to:
control energy delivery to a plurality of needles, the needles being configured to delivery energy from a location within a muscle layer of a stomach wall; and
cooperate with the plurality of needles to regulate energy delivery in a manner such that the energy delivered via the plurality of needles is substantially confined at the muscle layer.
